(19) United States
(12) Patent Application Publication
(10) Pub. No.: US 2023/0148940 A1
(43) Pub. Date: May 18, 2023

(54) CONCUSSION DETECTION AND DIAGNOSIS SYSTEM AND METHOD

(71) Applicant: Chiming Huang, Shawnee Mission, KS (US)

(72) Inventor: Chiming Huang, Shawnee Mission, KS (US)

(21) Appl. No.: 17/986,281

(22) Filed: Nov. 14, 2022

Related U.S. Application Data
(60) Provisional application No. 63/278,871, filed on Nov. 12, 2021.

(51) Int. Cl. A61B 5/397; A61B 5/00

(52) U.S. Cl. CPC A61B 5/397; A61B 5/6802

(57) ABSTRACT

This invention is a system and method for the detecting and identifying concussive events as well as potentially harmful sub-concussive events. The scientific and technological basis of this invention is the loss of muscle tone associated with a concussive event, also similar to the loss of muscle tone during REM sleep or the dream sleep. In addition, the invention also helps to monitor, identify, and document repetitive, sub-concussive head impact events.

### CROSS-REFERENCE TO RELATED APPLICATION

This application claims priority in U.S. Provisional Patent Application No. 63/278,871 Filed Nov. 12, 2021, which is incorporated herein by reference.

### BACKGROUND OF THE INVENTION

### 1. Field of the Invention

The present invention relates generally to a concussion solution and method for use thereof, and more specifically to a concussion detection and diagnosis system and method.

### 2. Description of the Related Art

Closed-head traumatic brain injury (TBI) is typically a result of the inappropriate and harmful brain movement. Forces acting on the body or the head generally accelerate the brain. High positive acceleration or negative acceleration may cause the brain to move with enough force or acceleration to cause brain injury. In addition, these accelerations set up transient pressure and strain gradients within the soft neuronal tissue of the brain. These gradients can sometimes bring about dramatic disruptions in neuronal metabolism and function at the cellular and molecular level without obvious or noticeable movement of the brain at the macroscopic level. Such disruptions may cause diffuse axonal injuries or DAI which is manifested at the cellular or sub-cellular level. Such cellular or sub-cellular disruptions may further trigger events at the molecular level such as opening of the blood-brain-barrier (BBB), additional glutamatergic neurotoxicity or excite-toxicity due to manifestations at the neurotransmitter level. The types of brain injury may also be categorized as blast TBI, concussive TBI, or mild TBI, etc. Blast TBI may be experienced by military or law enforcement personnel while on patrol or traveling in a vehicle. Concussions which are also synonymous with mild TBI or mTBI may be suffered by athletes in sports such as hockey, boxing, soccer, or American football. Mild TBI may be experienced by anyone suffering a fall, a vehicular accident, a bicycle accident, or the like.

The concussion threshold for an individual may change over time. The period may be long and span over many years during which development or maturation occurs in children and adolescents. Or it may be short, spanning over a matter of minutes or days as it is now known that a person is likely to be more vulnerable to a second concussion immediately after a concussion.

To help provide objective and quantitative diagnostics for TBI, the high-tech industry has delivered MEMS (MicroElectroMechanical Systems) sensors to monitor impact-induced head kinematics, an approach to inform the biomechanics of impact. Such systems may include one or more acceleration sensors or head-impact-measurement devices coupled to the head of a football player. The systems may further include a processing element which informs the potential of a given head impact to cause harm, namely whether the acceleration measured by the sensors exceeds a certain constant value believed to be a threshold beyond which a concussion to the player may occur.

However, data collected from MEMS sensors predicted concussions with an accuracy at chance level [Broglio S P, Schnebel B, Sosnoff J J, Shin S, Fend X, He X, Zimmerman J (2010) Biomechanical properties of concussions in high school football, Med Sci Sports Exerc. 42(10:2064-2071].

A systematic review concluded that modern MEMS technology categorically failed to detect TBI and had no clinical utility [O'Connor K L, Rowson S, Duma S M, et al. (2017) Head-impact-measurement devices: A systematic review. J Athl Train. 2017; 52(3):206-27.], citing that MEMS sensors have “. . . low specificity in predicting concussive injury, did not have the requisite sensitivity . . . have limited clinical utility.”

At present, there has not been available a system or method for a reliable and accurate detection and diagnosis of concussions. The need is particularly acute for detection and diagnosis in real time. The present invention has the advantage of being a reliable and accurate detector of concussions.

EMG (electromyograph) technology can monitor impact-induced loss of muscle tone (LoMT) by blunt force and thereby offer direct and immediate insight on the acute manifestation of concussions. EMG-based TBI sensor should be a far more superior approach as a wearable TBI sensor than MEMS sensors. Compared with MEMS TBI sensors, EMG-based TBI sensors should offer un-matched performance in sensitivity and specificity for the detection and diagnosis of TBI in real-time and in the field. The quantitative feature of EMG-based concussion detection further offers opportunities for continuous improvement via data training or machine learning.

The reason for the present invention being superior compared with all such high-tech MEMS technology is because such loss of muscle or LoMT occur reliably, quickly. LoMT happens to all muscles. And TBI-related LoMT is often a complete loss of muscle tone. Yet, the concept of monitoring EMG as a potential means for the detection of concussions has never been introduced, let alone utilized throughout the history of the development of MEMS technology for concussion detection.

This invention is a system and device that allow the detection of muscle tone as a diagnosis tool for concussions.

### BRIEF SUMMARY OF THE INVENTION

The present invention generally provides a system and method for the accurate detection and diagnosis of potential head injuries using a combination of hardware and software. The present invention comprises a system, device, and method to generally facilitate and validate the detection and diagnosis of potential head injuries.

The present invention employs a combination of hardware and software.

Related technology such as that disclosed in U.S. Pat. Nos. 8,961,440 and 9,226,707 for a “Device and System to Reduce Traumatic Brain Injury,” are owned by the same inventor as the present application and are incorporated herein by reference.

### BRIEF DESCRIPTION OF THE DRAWINGS

The drawings constitute a part of this specification and include exemplary embodiments of the present invention illustrating various objects and features thereof.

FIG. 1 is a series of four successive frames (33 msec apart) adapted from a series of high-speed video frames showing loss of muscle tone (LoMT) in boxers. A hit to the head can cause LoMT in lower limbs such that they become unable to support the weight of the boxer. Such LoMT in fact occurs to every muscle group in the entire skeletal muscular system.

FIG. 2 is a graph showing various data such as those derived from FIG. 1 as a function of time after impact. With the marker placed on the top of the head of the boxer, the boxer starts “going down” at ˜25 msec after impact (line B). The orange trace showed the same marker in a head hit that did not result in knockout (data not shown). No such “going down” is evident (line A).

FIG. 3 is a graph showing (data derived from FIG. 1) a time course of the loss of muscle tone (LoMT) during a knockout hit to the head from a boxing match. LoMT can occur quickly, typically within 30 msec of the impact. Note that both the glove of the boxer (line D) and the angle of the head (line C) started moving down starting at 35 msec after impact. In addition, indicating loss of muscle tone. In addition, the head angle (line C) started to move at a faster rate (marked by arrowhead) immediately after impact (line C, before 35 msec), indicating a reduction of head-and-neck inertia because of LoMT.

FIG. 4 is a diagram of an EMG recorded during the transition between normal sleep and REM sleep (rapid eye movement or dream sleep). The sudden (occurs within milliseconds) and dramatic loss (by a factor of 100 or more) of muscle tone (LoMT, starting at arrow and beyond) serves to prevent a person from active motor movement during the dream [Arrigoni E, Chen M C, Fuller P M (2016) The anatomical, cellular and synaptic basis of motor atonia during rapid eye movement sleep, J Physiol 594.19 pp5391-5414].

FIG. 5 is flowchart diagramming the major steps leading to a concussion utilizing conventional MEMS sensors for concussion in the priori art.

FIG. 6 is a flowchart diagramming the major steps leading to a concussion using the present embodiment.

FIG. 7s is a flowchart diagramming the steps taken in practicing an embodiment of the present invention.

### DETAILED DESCRIPTION OF THE PREFERRED EMBODIMENTS

### I. Introduction and Environment

As required, detailed aspects of the present invention are disclosed herein. However, it is to be understood that the disclosed aspects are merely exemplary of the invention, which may be embodied in various forms. Therefore, specific structural and functional details disclosed herein are not to be interpreted as limiting, but merely as a basis for the claims and as a representative basis for teaching one skilled in the art how to variously employ the present invention in virtually any appropriately detailed structure.

Certain terminology will be used in the following description for convenience in reference only and will not be limiting. For example, up, down, front, back, right and left refer to the invention as orientated in the view being referred to. The words “inwardly” and “outwardly” refer to directions toward and away from, respectively, the geometric center of the aspect being described and designated parts thereof. Forwardly and rearwardly are generally in reference to the direction of travel, if appropriate. Said terminology will include the words specifically mentioned, derivatives thereof and words of similar meaning.

II. Contrasting Prior Art MEMS with a Preferred Embodiment of the Present Invention

Concussions can have serious short- and long-term consequences including chronic traumatic encephalopathy (CTE), which is an evolving diagnosis and has no known cure [Omalu B I, DeKosky S T, Minster R L, Kamboh M I, Hamilton R L, Wecht C H (2005), Chronic traumatic encephalopathy in a National Football League player, Neurosurgery 57: 128-134; McKee A C, Cantu R C, Nowinski C J, et al. (2009) Chronic traumatic encephalopathy in athletes: progressive tauopathy after repetitive head injury, J Neuropathol Exp Neurol 68:709-735].

To stop the TBI progression and start treatment begins with diagnosis. The Executive Summary of the latest NIH Pediatric Concussion Workshop stated that there are more than 30 clinical or consensus definitions of concussion, hampering diagnostics and comparison across different studies [available at https://meetings.ninds.nih.gov/assets/Pediatric_Concussion_Workshop/NIH_Pediatric_Concussion_Workshop_Executive_Summary_revised.pdf ]. At present, accurate diagnosis in the field for concussions or mild traumatic brain injuries (TBI) is still challenging as such diagnosis relies heavily on the subjective impressions and decisions of individual physicians.

To help provide objective and quantitative diagnostics for TBI, the high-tech industry has delivered MEMS (Micro Electro Mechanical Systems) sensors to monitor impact-induced head kinematics, an approach to inform the biomechanics of impact with objectivity.

However, previously data collected from MEMS sensors predicted concussions with an accuracy at chance level [Broglio S P, Schnebel B, Sosnoff J J, Shin S, Fend X, He X, Zimmerman J (2010) Biomechanical properties of concussions in high school football, Med Sci Sports Exerc. 42(10:2064-2071].

A systematic review concluded that modern MEMS technology categorically failed to detect TBI and had no clinical utility [O'Connor K L, Rowson S, Duma S M, et al. (2017) Head-impact-measurement devices: A systematic review. J Athl Train. 2017; 52(3):206-27], citing that MEMS sensors have “. . . low specificity in predicting concussive injury, did not have the requisite sensitivity . . . have limited clinical utility.”

The reason for the difficulties in using MEMS technology to accurately identify concussive events largely lies in the many complex biomechanical and neurological steps between the initial head impact and the concussive damage to brain tissue. This complexity precludes the sure determination of a concussion solely based on the physical parameters of the impact force alone and causes the correlation to remain at chance level between MEMS data and the concussion. This problem is particularly acute in the range of force most commonly encountered in sports such as soccer and American Football. [Broglio S P, Schnebel B, Sosnoff J J, Shin S, Fend X, He X, Zimmerman J (2010) Biomechanical properties of concussions in high school football, Med Sci Sports Exerc. 42(11):2064-2071; O'Connor K L, Rowson S, Duma S M, et al. (2017) Head-impact-measurement devices: A systematic review. J Athl Train. 2017; 52(3):206-27].

EMG-based TBI technology can monitor impact-induced loss of muscle tone (LoMT) by blunt force and thereby offer direct and immediate insight on the acute manifestation of concussions. Because LoMT is only present during and after a concussive event, this approach is a superior approach in sensitivity and specificity for the detection and diagnosis of TBI in the field. (As an example, the loss of muscle tone in boxing matches and MMA fights is often only present for a few seconds and observable immediately after a knockout hit. However transient, knockout or knock down is highly sensitive and specific to the event.)

Yet at present, in many sports such as football or soccer, muscle tone or LoMT does not occupy a prominent position among acute signs of concussions. This is likely because such LoMT is transient, often lasting only seconds. [e.g., Mayo Clinic (2020) https://www.mayoclinic.org/diseases-conditions/concussion/symptoms-causes/syc-20355594 , last updated 2-20-2020, last visited 10-4-2021].

From observations in preliminary studies on boxing matches, we have discovered that LoMT is usually immediately detectible after an impact to the head, but can be quite transient in many KO/TKO decisions. It appears quickly (in milliseconds) and typically lasts only seconds.

Often in boxing matches, there is one final head blow which prompts the referee to stop the fight followed with a KO or TKO (knockout or technical knockout) decision. A consistent finding in our preliminary studies, we observed an immediate and transient LoMT which always precedes the KO or TKO decision. We examined the characteristics of LoMT in such KO or TKO decisions, including the time course and the scope of LoMT. For timing, we asked how quickly the LoMT can occur after a head hit. For scope, we asked how severe and how global the LoMT is.

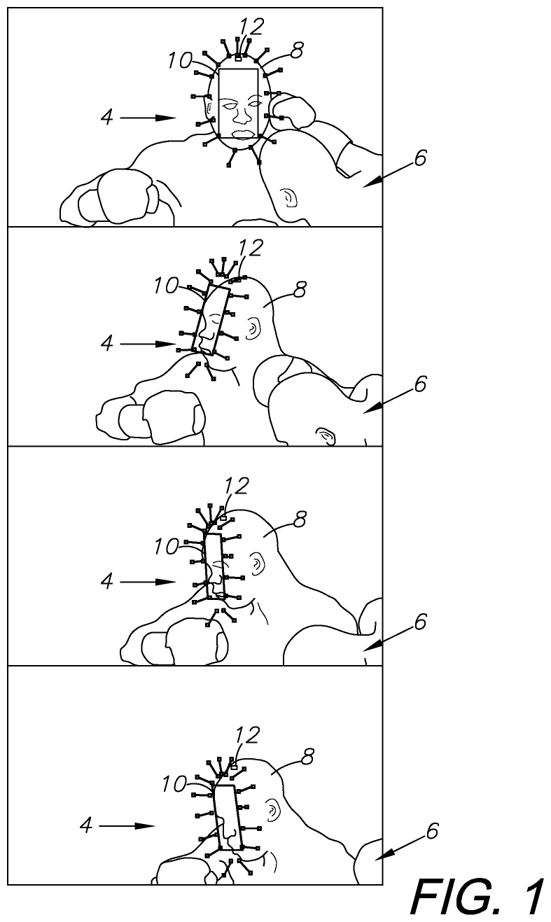

First, LoMT affected the muscle tone in the lower limbs. For example, to visualize how the leg muscles keep a boxer standing, we tracked the top of the boxer's head, as shown in the series of images in FIG. 1. FIG. 1 shows a first boxer 4 and a second boxer 6, where the first boxer was being struck with a force from the second boxer. Just over 100 milliseconds separate the top frame from the bottom frame. In the bottom frame, barely 100 msec or one tenth of a second after the hit, the first boxer's 4 head dropped significantly. This assumes and requires that the camera position to not fundamentally move between the images. Tracking software can be used to monitor the position of the first boxer's 4 head 8, as indicated by the tracking box 10.

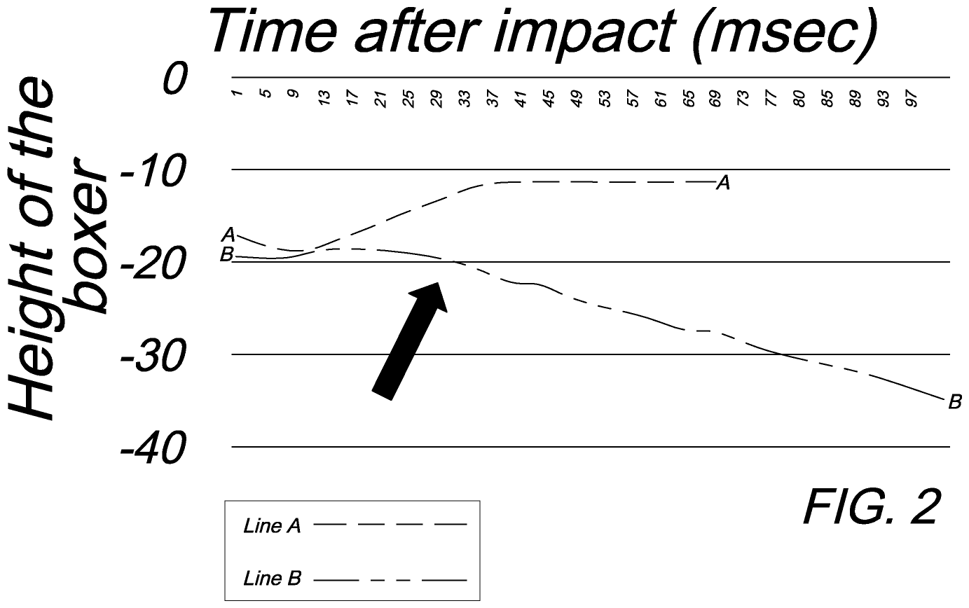

To further examine the observations made in FIG. 1 quantitatively, a tracking marker 12 was placed on the top of the first boxer's 4 head 8 and a HD (high definition) video was filmed at high speed (˜1 kHz) in order to monitor quantitatively the height of the marker as a function of time. FIG. 2 shows that in a head hit that did not lead to a KO call, the boxer's height stayed unchanged after the hit (Line A). However, in a KO hit, the boxer starts going toward the canvas ˜30 msec after the head hit (Line B).

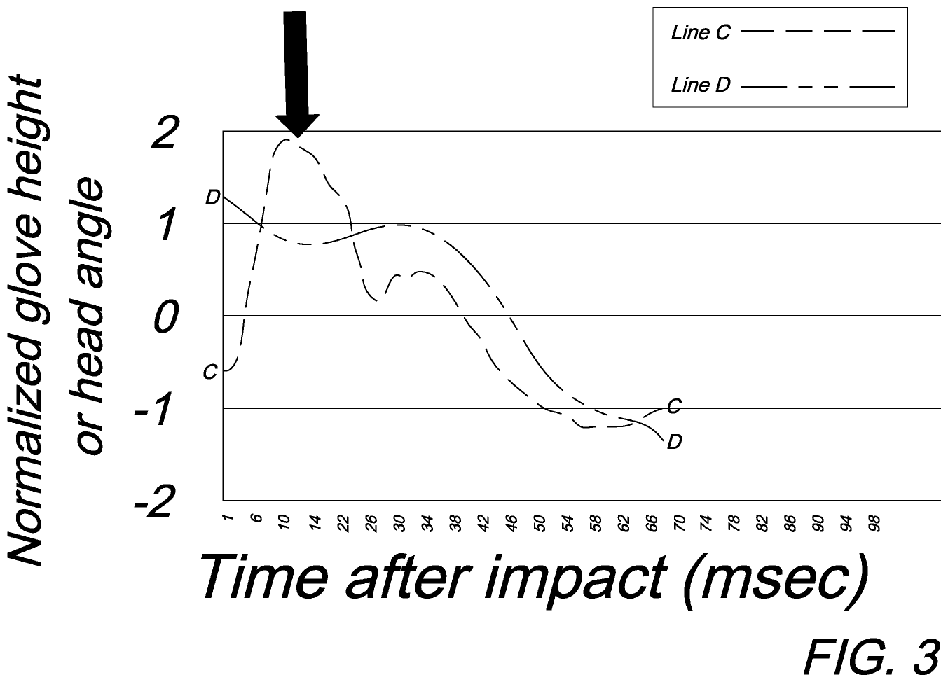

FIG. 3 shows LoMT in the upper limb and the head-and-neck musculature. Boxers are trained to keep their fists high (e.g., at eye level or at least above the shoulder). However, a fighter often lowered his fists as if the arms were suddenly unable to lift his fists (Line D). Head angular rotations after a hit were also measured. The head angle (Line C) initially went through a series of changes as a direct result of the head hit. For example, the first hump in Line C represents the head moving by the hit and then snapping back (within the first 35 msec). Then the head angle stays there for some time (from 35 to 50 msec). Curious enough, the head angle started to take off again after 50 msec, when the fist had left the head long ago. This is due to the LoMT in the head-and-neck muscle, which decreases the effective mass of the head. It is clear that LoMT renders the head-and-neck muscles no longer able to resist any residual force and thus making the head movement more obvious. Moreover, Line C and Line D appear to take off at about the same time—roughly 50 msec after the hit, indicating LoMT clearly.

There was LoMT detected in the muscles of the torso as well. A football player can often be seen to fall “limp” as if superficial, deep, and intrinsic back muscles are all without tone

A boxer often grimaced as he experienced a painful blow to the body, such as near the lower part of the rib cage. Grimacing was never observed after a hit to the head in the KO or TKO cases examined. A hit to the head may be not as “painful” as a hit to the rib cage, but it is more likely that the facial musculature may have lost its muscle tone.

The loss of muscle tone in KO events can be severe or nearly complete, particularly in cases involving a loss of consciousness (LOC), even the LOC is partial and lasted only seconds. In these cases, the severe loss of use of skeletal musculature resembled a sudden attack in patients of cataplexy. Such LoMT can best be described as an active person transformed into an inanimate object, followed by free fall in gravity accompanied by flaccidity or paralysis

The speed, the scope, and the severity of the muscle tone loss in LoMT is not consistent with a local, loss-of-function mechanism. In addition, intuitively, it is not at all clear why the legs should be affected when the hit was not even close to where the legs are.

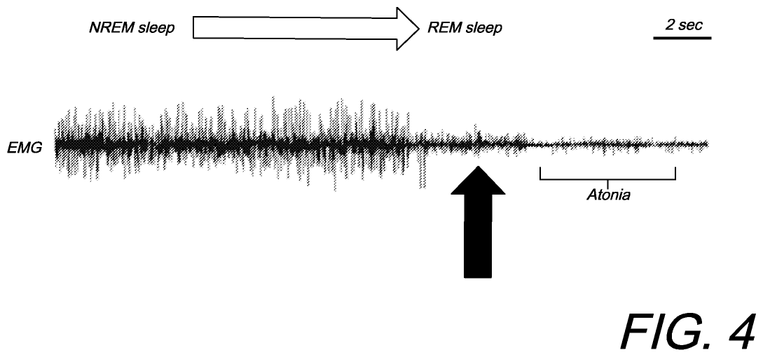

However, beyond the intuitive level and into neuroscience, observations on LoMT suggest that the mechanisms generating LoMT is congruent with an active mechanism mediated by the central nervous system to shut down the muscle tone actively and globally. Similarly, severe loss of muscle tone affecting the skeletal musculature globally can be seen during REM sleep [FIG. 4, Arrigoni E, Chen M C, Fuller P M (2016). The anatomical, cellular and synaptic basis of motor atonia during rapid eye movement sleep, J Physiol 594.19 pp5391-5414].

The conjecture that impact-induced LoMT reflects a mechanism originated within the central nervous system also receives support from observations on the facial musculature of boxers in KO. The tone in facial musculature is mediated via brainstem trigeminal and facial centers. With the facial musculature behaving like the rest of the skeletal musculature (under the control of C2 to C4 segments of the spinal cord), such LoMT is likely to be controlled by a more rostral structure in the central nervous system that can influence cervical segments of the spinal cord as well as the brainstem. Therefore, based on plausible neural mechanisms of LoMT, utilizing EMG-based TBI sensor technology monitoring LoMT can offer unique insights on such brain structures in real-time during concussions.

To summarize our preliminary studies of LoMT up to this point, LoMT manifests immediately following a KO head hit. EMG-based TBI sensors can monitor such LoMT. Because LoMT stems from a neural event, which reflects the resultant mTBI, and not a biomechanical event, which may or may not be causing mTBI (e.g., as in MEMS sensors which monitors head accelerations), EMG-based TBI sensors should be a promising approach with high sensitivity and specificity for detecting TBI.

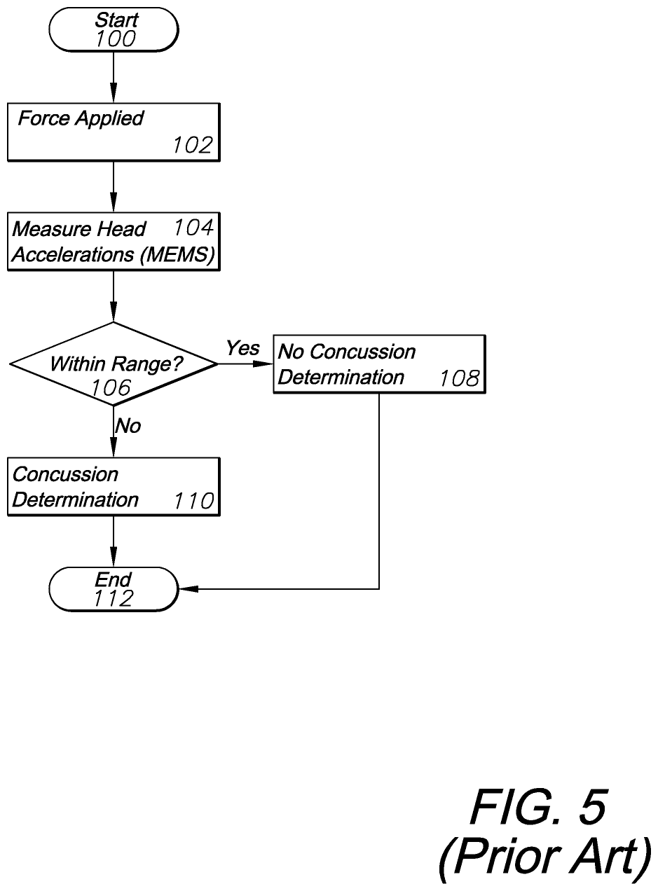

FIG. 5 shows where the detection of LoMT would appear in a prior art, broadly utilizing traditional MEMS sensor system. The process starts at 100 where force is applied to the subjects' head at 102, after which the system measures the head acceleration using a traditional MEMS system sensor at 104. If that result is within an acceptable range at 106, no concussion is determined, and the process ends at 112. The present invention would aim at measuring the LoMT at and after steps 108 and 110 rather than focusing on the previous MEMS concussion detection process.

As discussed previously, conventional MEMS sensors collect data on head accelerations. These sensors cannot accurately detect concussions because head accelerations may or may not cause concussions and are not clinically useful [Broglio S P, Schnebel B, Sosnoff J J, Shin S, Fend X, He X, Zimmerman J (2010) Biomechanical properties of concussions in high school football, Med Sci Sports Exerc. 42(11):2064-2071; O'Connor K L, Rowson S, Duma S M, et al. (2017) Head-impact-measurement devices: A systematic review. J Athl Train. 2017; 52(3):206-27].

The sensor of the present invention detects events more down-stream than events detected by the conventional MEMS sensors. The sensor detects LoMT, which only occur with a concussion. This is the major reason why the sensor in the present invention is far more reliable than the conventional MEMS sensors for concussion detection. This reliability comes from the high degree of specificity and sensitivity of the EMG-based concussion detection compared with the MEMS approach.

Indeed, the EMG-based TBI sensors of the present invention detect effects of concussions by focusing on the aftermath of concussions such as LoMT, which is due to neural mechanisms as a result of the head-impact. As explained below, the sensors of the present invention monitor the characteristics of blunt force impact-induced loss of muscle tone, which can only occur after a concussion, which is not described in FIG. 5 although such LoMT always manifests immediately as a direct consequence of such TBI. The EMG-based sensors of the present invention therefore provide objective, quantitative, and diagnostic data for TBI in real time with a high degree of sensitivity and specificity.

It is almost certain that concussive events are graded events. In other words, among concussive events, some will be associated with more clinically serious consequences than others. In more serious head-impact events, for example, long-term coma can lead to LoMT that persist for days, weeks, and potentially into months. In less severe concussions, such LoMT may last only seconds. There may even be modifications of EMG in sub-concussive events. This area is currently unexplored.

It is also conceivable that some extent of LoMT may also manifest in sub-concussive head-impact events. Therefore, it stands to reason that it may be productive to examine and explore the length of LoMT (e.g., FIG. 4) as an index for grading the severity of a head-impact event—all the way from sub-concussive head-impact events to concussions. Due to the quantitative nature of the data obtained via EMG-based sensors, the present invention would be of utility in grading the severity of concussive events.

It is possible to identify and similarly quantify the occurrence of repetitive, harmful, sub-concussive head impact events utilizing the present invention. As previously stated, it is almost certain that these sub-concussive events are also graded events. For example, it is almost certain that there will be graded LoMT which is absent in head movements that are voluntary and harmless, and which begins to manifest in impact-induced head movements in sub-concussive incidences right up to the threshold of concussions. Therefore, the present invention would be of utility in also grading the harmful potential of repetitive, sub-concussive head-impact events.

In addition, patients of PTSD and TBI often share overlapping symptoms which create difficulties in treatment decisions. The approach using LoMT to detect and identify incidences of TBI on record may pave the way for a potential differential diagnosis of PTSD and TBI.

III. Implementation and methods of the Concussion Detection System 2

Raw data sampling in the time dimension: EMG signals are sampled as voltage values over time. Such signals from one region of the skeletal muscle (e.g., tibialis anterior) will be sampled by a miniaturized, wearable sensor 20 including a surface electrode 22 for EMG and digitized, for the purpose of sampling frequency, at ˜5 kHz with an adjustable range between 2-10 kHz.

EMG signals 24 from surface electrodes 22 reflect the action potentials of muscle cells. As such action potentials from individual muscle cells are typically 0.5 to 1.5 msec in duration, the major power of EMG from surface electrodes will center about 1 kHz and taper off on both directions of the frequency axis. The range of sampling frequency descried above will therefore allow the present invention to catch the overwhelming bulk of the EMG activities which are reflections of the action potentials of skeletal muscles.

Raw data sampling in the voltage dimension: Each one of the timed data points on voltage will contain ˜10 bits of information with an adjustable range of 8 to 16 bits.

Raw data processing: The raw EMG data will be initially stored in a working memory in multiples of 10,000 to 15,000 data points. At the sampling rate of 2-10 kHz, the time period covered can be flexible and between 1 to 7.5 seconds. The objective is to chop or parcel the continuous EMG data stream into memory multiples with each of the multiples holding several seconds (e.g., 2-3 seconds) of EMG data. This parceling process can operate with an adjustable range so that each of these parcels or epochs contains anywhere from 1 to 7.5 seconds of EMG data.

Raw data storage: Once these operating parameters are set, the EMG data will be stored as successive files (order by time) of fixed length. Each such files will contain approximately 1 to 7.5 seconds (on average ˜2-3 seconds) of raw EMG data, with identifiers including date, time, user ID, and other identifiers.

Data reduction: The data in each of the files will be processed to extract and thereby generate a few key numbers describing the overall characteristics of EMG data. For example, the voltage values in each of the multiple EMG epoch files can be integrated and reduced to a number reflecting the mean EMG amplitude over the entire period (˜2-3 seconds) of the epoch. This allows the option of storing just the overall characteristics of the EMG data within a time epoch of several seconds rather than the entire raw records of EMG withing the same time epoch, thereby dramatically reducing the memory required for data storage.

Data analysis: Data analysis will be focused to identify the sudden transition between normal, chaotic, and relatively high level of EMG activity to abnormally and consistently low levels (reduced by a factor of 10 or more) of EMG over a significantly longer periods of time (e.g., one to several seconds, see the transition into muscular atonia in FIG. 4).

Example: Identifying abnormally and consistently low levels of EMG (reduced by a factor of 10 or more) over a significantly longer periods of time (e.g., one to several seconds) by comparing the level of EMG in the current epoch with the statistical sample consisting of the most recent epochs of EMG data, for example, the last 10 time epochs, powered by an on-the-fly Student t test.

Data management: Time constant for data lumping: In dividing the continuous stream of EMG data into discrete epochs of EMG data and further providing a single number to represent the average amplitude of EMG activity in individual epochs, the following considerations must be considered:
a. The duration of the epoch should not be significantly greater than the time it takes for LoMT to occur after a concussive head-impact event. Not following this consideration will cause information to be lost on the precise time when concussion takes place.
b. The duration of the epoch should not be significantly less than the time it takes for LoMT to occur after a concussive head-impact event. Not following this consideration will cause the data management to be needlessly cumbersome.
c. Therefore, it is stipulated that the EMG epoch should be less than 2 seconds but significantly longer than 10 msec.
d. In general applications, and according to our preliminary studies on knockouts in boxing matches and MMA fights, 30 msec is a good start; however, 100-300 msec would work as well. In summary, although we have described the principles of setting the value of these operating parameters, the exact and optimal values for these parameters can be set pending more available data.
e. Focus on the detection of sudden LoMT, flag out the timing, and sound alarm.

Data Storage: Highly analyzed EMG data will be uploaded to a remote server for storage. Because the system 2 has processed the EMG data via integration, such data will be far more compact than the raw EMG data as voltage over time. This will facilitate storage, allowing us to keep abreast of the EMG over a much longer period of time (e.g., later use for artificial intelligence and machine learning).

Another implementation technique to enhance the success in detecting LoMT can involve the simultaneous recording of EMG activities from more than one surface EMG electrodes. Because impact induced LoMT occurs to virtually all skeletal musculatures, the system can employ the strategy of correlation or coincidence detection to determine whether signs of LoMT occur at nearly the same time and can be identified as such by examining the EMG activities from two different recording sites. This application or embodiment will dramatically decrease the probability of false positives as well as false negatives, thereby improving the sensitivity as well as specificity.

It is particularly advantageous to monitor EMG from two antagonistic muscles from opposite side of a joint in order to detect the simultaneous loss of muscle tone in these antagonistic muscles (an example will be the triceps and the biceps). This approach will dramatically increase the reliability (sensitivity and specificity) of TBI detection as the simultaneous loss of muscle tone are highly unlikely to occur in physiological-relevant situations.

The strategy of coincidence analysis may be particularly useful in identifying the LoMT associated with sub-concussive head impact events in which the transition from normal EMG to muscular atonia may be not as clear-cut as shown in FIG. 4.

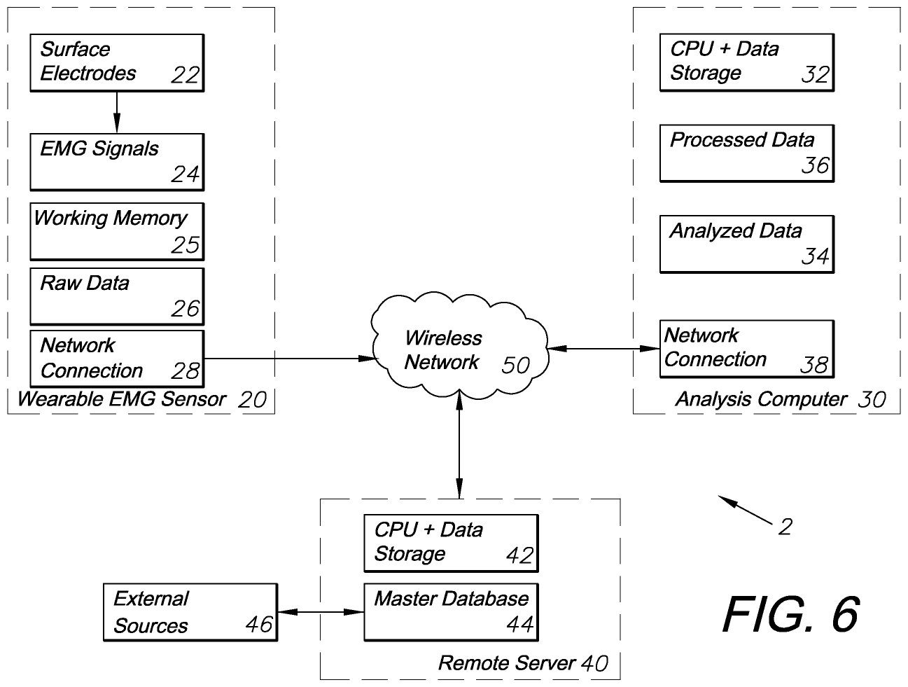

FIG. 6 is a block diagram showing the relationship between elements of the present invention. The primary element of the invention is the Wearable EMG Sensor 20 which includes at least one surface electrode 22 which produces EMG signals 24 which are used to generate raw data 26 usable to detect a concussion based upon LoMT, stored within a working memory 25. A network connection 28, such as a wireless protocol, allows the sensor 20 to send the raw data through a wireless network 50 to an analysis computer 30.

The analysis computer 30 includes a CPU and data storage 32, the analyzed data 34 which is created from the raw data 26 received from the sensor 20. This processed data 36 is stored as described above within the data storage 32 of the analysis computer 30, whereafter the CPU analyzes the data 42 to generate analyzed data 34. Through the analysis computer 30 network connection 38, this analyzed data can be sent to be stored at the remote server 40 as described above. The analyzed data would be incorporated into the master database 44 which also may include external data sources 46 to further enhance machine learning and predictive analysis of concussions.

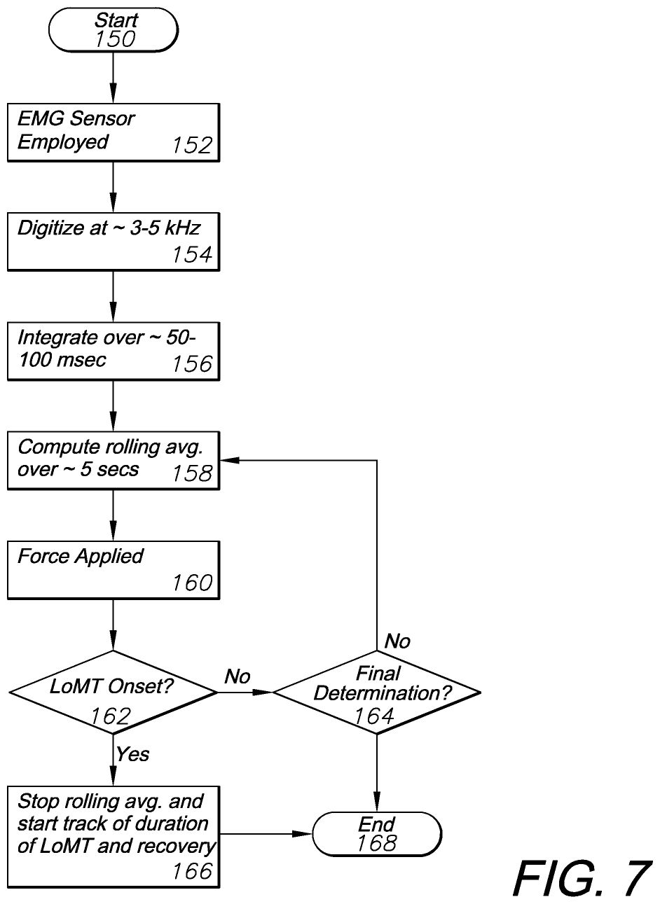

FIG. 7 is a flow chart describing the steps taken in implementing the components in one version of the system and device of the present invention.

The process is started at 150 where the EMG sensor 20 is employed 152 and placed on a subject. The EMG sensor is digitized at approximately 3-5 kHz at 154 and integrated over approximately 50-100 milliseconds at 156. The analysis computer 30 will compute a rolling average over five seconds, and the sensor 20 will detect whether there is LoMT onset at 160. If not, the process can continue monitoring, computing rolling averages over five seconds at a time at 158 until LoMT onset is detected at 160, after which the system stops the rolling average and begins instead to track the duration of LoMT and recovery at 164, after which the process ends at 166. If LoMT onset is never detected at 164 and the monitoring is no longer required at 162, then the process ends at 168.

To review the discussion on implementation and methods up to this point, the system shown in FIG. 6 and the steps shown in FIG. 7 will allow the system 2 to perform the following tasks. (1) Collect and sample EMG data. (2) Average EMG data in ˜100 msec and get a number; in this case approximately ten numbers for each second of elapsed time. (3) Keep ˜30-50 of these numbers in memory, in this case covering a period of 3 to 5 seconds. Obtain mean and SD by considering each ˜100 msec of EMG data as a single sample. (4) Always ask whether the newest number is significantly different from the historical data by ˜3 or more SD on the small side. Use rolling average so the average and SD always reflect the most recent ˜30-50 data points. (5) Sound alarm if the newest data is ˜3 or more SD below the mean. (6) Keep sampling data. Stop the rolling average and fixate on the one average that help to net the outlier.

Monitor recovery: Keep testing the newest data same way without rolling average. And continue flagging the alarm. Also keep track of the number of these low outliers. Keep doing that until the “newest” climbs back to within two standard deviations; one standard deviation; etc.

The duration LoMT should be directly proportional to the severity of the concussive event. Therefore, monitoring the length of time it takes for EMG to recover to normal levels is very important and can reveal data on the severity of the concussive event.

In addition, the EMG data in storage will be a data source with which the EMG-based TBI sensor can machine-learn and therefore become “smart” in identifying concussive events with a personal touch, thereby empower the TBI sensor to be of utility in terms of personalized medicine or personalized healthcare.

This approach generates quantitative data with a high degree of objectivity, in real time, and in the field. Our invention is therefore a new, wearable, EMG-based TBI sensor that is capable of generating data that is sensitive, specific, objective, quantitative, accurate, and fast for TBI diagnosis. The sensor and its related support algorithms are easy as well as cost-effective to implement. Since the LoMT in boxers experiencing KO or TKO is immediate and severe while involving muscles in virtually all dermatomes, its neural mechanisms are likely to be related to the muscular atonia in REM sleep. LoMT for TBI diagnosis is therefore a well formulated hypothesis on sound scientific rationale.

Having thus described the invention, what is claimed as new and desired to be secured by Letters Patent is:

1. A system for detecting and identifying a concussive event, the system comprising:
a wearable sensor device for generating electromyographic (EMG) data from a body;
a computing device comprising a CPU and data storage, said computing device in communication with said wearable sensor such that said computing devices is configured to said wearable sensor;
wherein said wearable sensor communicates a loss of muscle tone (LoMT) indicative of a concussive event to said computing device as processed data;
said computing device configured to analyze said processed data against a predetermined dataset correlating LoMT and previous concussive determinations thereby resulting in the detection of a concussive event; and
said computing device further configured to generate a warning upon the detection and identification of said concussive event.

2. A method for the detection and identification of concussive events, the method comprising:
sensing and recording EMG data via a wearable sensor placed on a body;
of processing in real-time said EMG data with a computing device comprising a CPU and data storage, thereby identifying episodes of sudden and significant loss of muscle tone (LoMT) indicative of a concussive event and generating results; and
storing said results for the purpose of aiding concussion diagnosis.

3. A method for the detection and identification of concussive events, the method comprising:
sensing and recording EMG data via a wearable sensor placed on a body;
processing in real-time said EMG data and identifying episodes of sudden and significant loss of muscle tone (LoMT) indicative of a harmful but sub-concussive event; and
storing the results of said data processing on episodes of LoMT for the purpose of aiding the diagnosis of a harmful but sub-concussive event.
